(19) United States
(12) Patent Application Publication
(10) Pub. No.: US 2004/0214310 A1
(43) Pub. Date: Oct. 28, 2004

(54) Apparatus and method for array alignment

(76) Inventors: Russell A. Parker, San Jose, CA (US); Jacqueline Tso, Los Gatos, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/424,175

(22) Filed: Apr. 25, 2003

(51) Int. Cl. C12M 1/34

(52) U.S. Cl. 435/287.2; 435/288.3

(57) ABSTRACT

An apparatus and method for array alignment. The apparatus employs a standoff that is adjacent to a gasket and that may be attached to a first substrate or a second substrate. The standoff is larger in height than the gasket that holds an array solution. The larger height of the standoff relative to the gasket helps during the alignment of the first substrate and the second substrate and prevents the spreading or spillage of the array solution when attempts are made to contact the first substrate to the second substrate. Methods of making and aligning the array hybridization apparatus are also disclosed.

### FIELD OF THE INVENTION

The invention relates to the field of micro arrays, and more particularly to a standoff for use in microarray processing, loading and alignment.

### BACKGROUND OF THE INVENTION

Polynucleotide arrays (such as DNA or RNA arrays) are known and are used, for example, as diagnostic or screening tools. Such arrays include regions of usually different sequence polynucleotides arranged in a predetermined configuration on a substrate. These regions (sometimes referenced as “features”) are positioned at respective locations (“addresses”) on the substrate. In use, the arrays, when exposed to a sample, will exhibit an observed binding or hybridization pattern. This binding pattern can be detected upon interrogating the array. For example, all polynucleotide targets (for example, DNA) in the sample can be labeled with a suitable label (such as a fluorescent dye), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Biopolymer arrays can be fabricated by depositing previously obtained biopolymers (such as from synthesis or natural sources) onto a substrate, or by in situ synthesis methods. Methods of depositing obtained biopolymers include dispensing droplets to a substrate from dispensers such as pin or capillaries (such as described in U.S. Pat. No. 5,807,522) or such as pulse-jets (such as a piezoelectric inkjet head, as described in PCT publications WO 95/25116 and WO 98/41531, and elsewhere). For in situ fabrication methods, multiple different reagent droplets are deposited from drop dispensers at a given target location in order to form the final feature (hence a probe of the feature is synthesized on the array substrate). The in situ fabrication methods include those described in U.S. Pat. No. 5,449,754 for synthesizing peptide arrays, and described in WO 98/41531 and the references cited therein for polynucleotides. The in situ method for fabricating a polynucleotide array typically follows, at each of the multiple different addresses at which features are to be formed, the same conventional iterative sequence used in forming polynucleotides from nucleoside reagents on a support by methods of known chemistry. This iterative sequence is as follows: (a) coupling a selected nucleoside through a phosphite linkage to a functionalized support in the first iteration, or a nucleoside bound to the substrate (i.e. the nucleoside-modified substrate) in subsequent iterations; (b) optionally, but preferably, blocking unreacted hydroxyl groups on the substrate bound nucleoside; (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in a known manner.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, and elsewhere.

The present invention also provides methods for using one or more arrays protected by a housing with an array assay such as a hybridization assay or any other analogous binding interaction assay. Generally in these binding assays, a sample suspected of including an analyte of interest, i.e., a target molecule is contacted with an array under conditions sufficient for the analyte target in the sample to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system, e.g., an isotopic or fluorescent label or the like present on the analyte, as described above. The presence of the analyte in the sample is then deduced from the detection of binding complexes on the substrate surface.

As mentioned above, arrays held in a subject array holder may be used in a variety of array-based assays, where hybridization reactions will be used herein for exemplary purposes only, and is not intended to limit the scope of the invention. In hybridization assays, a sample of target analyte such as target nucleic acids is first prepared, where preparation may include labeling of the target nucleic acids with a label, e.g., with a member of signal producing system, and the sample is then contacted with the array, e.g., a nucleic acid array, under hybridization conditions, whereby complexes are formed between target analytes such as nucleic acids that are complementary to probe sequences attached to the array surface. The presence of hybridized complexes is then detected as described above. Specific hybridization assays of interest that may be practiced using the subject methods include: gene discovery assays, differential gene expression analysis assays; nucleic acid sequencing assays, and the like. Patent applications describing methods of using arrays in various applications include: WO 95/21265; WO 96/31622; WO 97/10365; WO 97/27317; EP 373 203; and EP 785 280 and U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of the U.S. Patents which are herein incorporated by reference.

A number of problems exist in the art regarding loading and use of these arrays. Loading arrays into a hybridization chamber is a first problem. After the backing has been filled to the appropriate level, the array must be gently placed over the backing with no tilt to the glass or substrate as it approaches the backing with the liquid. In addition, in certain instances the backing or array may contact prematurely and cause spreading and loss of the sample volume held by the gasket on the backing. Array loading is often difficult to do correctly without loss of sample or inaccurate positioning. The current assembly and art offer little help in solving this problem. Of the spacers and gaskets available or used in the art, none prevent the smearing and sample loss problems. Secondly, in certain instances it may be preferable to employ pre-designed gaskets of particular size and shape for separating arrays and solutions. In addition, certain larger gaskets may also be employed that can contain larger array hybridizations and solutions. It would, therefore, be desirable to provide an array loading device that satisfies these and other needs. These problems and other are addressed by the present invention.

### SUMMARY OF THE INVENTION

The invention provides an array hybridization apparatus and method of making and aligning the same. The array hybridization apparatus comprises a first substrate for holding an array, a second substrate opposite the first substrate for acting as a backing for the array hybridization apparatus, and a standoff interposed between the first substrate and the second substrate. An array hybridization apparatus may be easily aligned when at least one of the substrates contacts the standoff. Since the standoff comprises deformable or substantially deformable materials it will not interfere with the construction of the hybridization assembly when the first substrate contacts the second substrate and gaskets. The invention also provides a method for making an array hybridization apparatus.

### BRIEF DESCRIPTION OF THE DRAWINGS

Embodiments of the invention will now be described with reference to the drawings, in which:

FIG. 1 illustrates a first substrate carrying an array, of the invention;

FIG. 2 is an enlarged view of a portion of FIG. 1 showing ideal spots or features;

FIG. 3 is an enlarged illustration of a portion of the substrate shown in FIG.2.

FIG. 4A is a side elevation view of a first embodiment of the present invention;

FIG. 4B shows a perspective view of a second embodiment of the present invention;

FIG. 4C shows a third embodiment of the invention;

FIG. 5 shows a cross sectional view of the present invention;

FIGS. 6A and 6B show enlarged cross sectional areas of FIG. 5 and how the invention operates.

### DETAILED DESCRIPTION OF THE INVENTION

Before describing the invention in detail, it must be noted that, as used in this specification and the appended claims, the singular forms “a,” “an,” and “the” include plural referents unless the context clearly dictates otherwise. Thus, for example, reference to “a gasket” includes more than one “gasket”. Reference to a “standoff” or “substrate” includes more than one “standoff” or “substrate”. In describing and claiming the present invention, the following terminology will be used in accordance with the definitions set out below.

A “biopolymer” is a polymer of one or more types of repeating units. Biopolymers are typically found in biological systems (although they may be made synthetically) and particularly include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A “peptide” is used to refer to an amino acid multimer of any length (for example, more than 10, 10 to 100, or more amino acid units). A biomonomer fluid or biopolymer fluid references a liquid containing either a biomonomer or biopolymer, respectively (typically in solution).

A “set” or “sub-set” of any item (for example, a set of features) may contain one or more than one of the item (for example, a set of clamp members may contain one or more such members). An “array”, unless a contrary intention appears, includes any one, two or three dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one that is to be evaluated by the other (thus, either one could be an unknown mixture of polynucleotides to be evaluated by binding with the other). An “array layout” refers collectively to one or more characteristics of the features, such as feature positioning, one or more feature dimensions, and some indication of a moiety at a given location. “Hybridizing” and “binding”, with respect to polynucleotides, are used interchangeably. When one item is indicated as being “remote” from another, this is referenced that the two items are at least in different buildings, and may be at least one mile, ten miles, or at least one hundred miles apart.

The term “adjacent” or “adjacent to” refers to a component or element that is near, next to or adjoining. For instance, a gasket may be adjacent to a standoff.

The term “substantially deformable”, “compressible” or “deformable” shall all have a similar meaning.

The term “first substrate” refers to any number of materials having at least one planar surface capable of contacting a gasket or standoff. The term shall be broad based to include substrates, polymeric materials, silica based materials, plastics etc.. It's important that the “first substrate” maintain a certain amount of rigidity to compress or deform the gasket and contact the standoff. In certain instances a “first substrate” will be transparent to allow light to pass through its medium. However, this is not required. Also, the “first substrate” must be capable in certain instances to allow for the mounting or construction of an array on its surface, although in certain cases this will not be required if the array is constructed on a separate surface.

The term “second substrate” shall refer to any number of materials well know in the art that are capable of acting as a mounting medium.

It will also be appreciated that throughout the present application, that words such as “front”, “rear”, “back”, “leading”, “trailing”, “top”, “upper”, and “lower”, are all used in a relative sense only. “Fluid” is used herein to reference a liquid. Reference to a singular item, includes the possibility that there are plural of the same items present. Furthermore, when one thing is “slid” or “moved” or the like, with respect to another, this implies relative motion only such that either thing or both might actually be moved in relation to the other.

All patents and other cited references are incorporated into this application by reference.

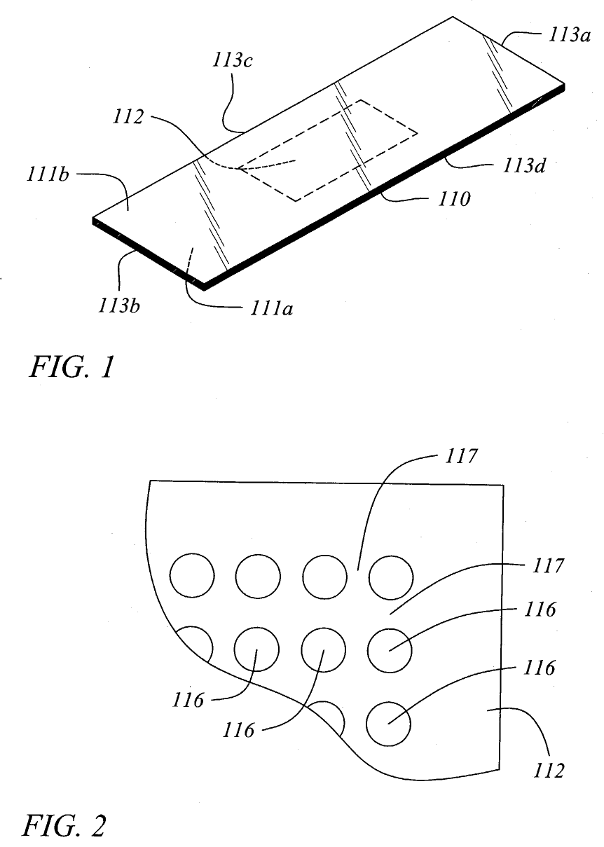

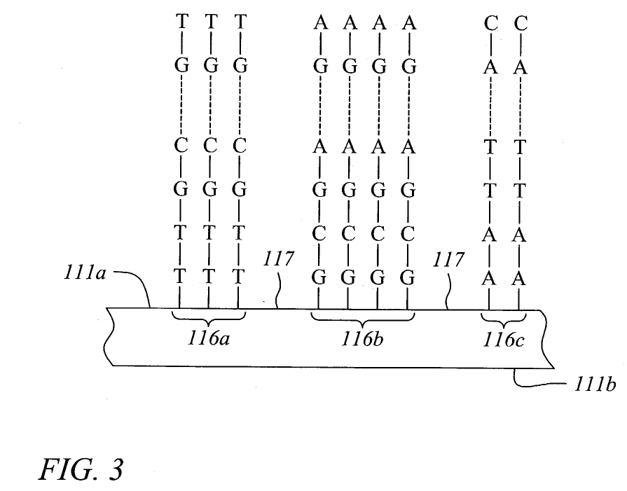

Referring first to FIGS. 1-3, typically the methods and apparatus of the present invention generate or use a contiguous planar first substrate 110 carrying an array 112 disposed on a rear surface 111a. It will be appreciated though, that more than one array (any of which are the same or different) may be present on the rear surface 111a, with or without spacing between such arrays. Note that one or more of the arrays 112 together will cover the substantial regions of the rear surface 111a, with regions of the rear surface 111a adjacent to the opposed sides 113c, 113d and the leading end 113a and the trailing end 113b of the first substrate 110. A front surface 111b of the first substrate 110 does not carry any of the arrays 112. Each of the arrays 112 can be designed for testing against any type of sample, whether a trial sample, reference sample, a combination of them, or a known mixture of polynucleotides (in which latter case the arrays may be composed of features carrying unknown sequences to be evaluated). The first substrate 110 may be of any shape, and any holder used with it adapted accordingly, although the first substrate 110 will typically be rectangular in practice. The array 112 contains multiple spots or features 116 of biopolymers in the form of polynucleotides. A typical array may contain from more than ten, more than one hundred, more than one thousand or ten thousand features, or even more than from one hundred thousand features. All of the features 116 may be different, or some or all could be the same. In the case where the array 112 is formed by the conventional in situ or deposition of previously obtained moieties, as described above, by depositing for each feature at least one droplet of reagent such as by using a pulse jet such as an inkjet type head, interfeature areas 117 will typically be present which do not carry any polynucleotide. It will be appreciated though, that the interfeature areas 117 could be of various sizes and configurations. Each feature carries a predetermined polynucleotide (which includes the possibility of mixtures of polynucleotides). As per usual, A, C, G, T represent the usual nucleotides. It will be understood that there may be a linker molecule (not shown) of any known types between the rear surface 111a and the first nucleotide.

The first substrate 110 may also carry on the front surface 111b, an identification code in the form of a bar code 115 printed on an opaque substrate in the form of a paper label attached by adhesive to the front side 111a (not shown in FIGS.). By “opaque” in this context is referenced that the means used to read the bar code 115 (typically a laser beam) can not read the bar code 115 through the label without reading errors. Typically this means that less than 60% or even less than 50%, 30%, 20% or 10% of the signal from the code passes through the substrate. The bar code 115 contains an identification of the array 112 and either contains or is associated with, array layout or layout error information.

For the purposes of the discussions below, it will be assumed (unless the contrary is indicated) that the array 112 is a polynucleotide array formed by the deposition of previously obtained polynucleotides using pulse jet deposition units. However, it will be appreciated that an array of other polymers or chemical moieties generally, whether formed by multiple cycles in situ methods adding one or more monomers per cycle, or deposition of previously obtained moieties, or by other methods, may be present instead.

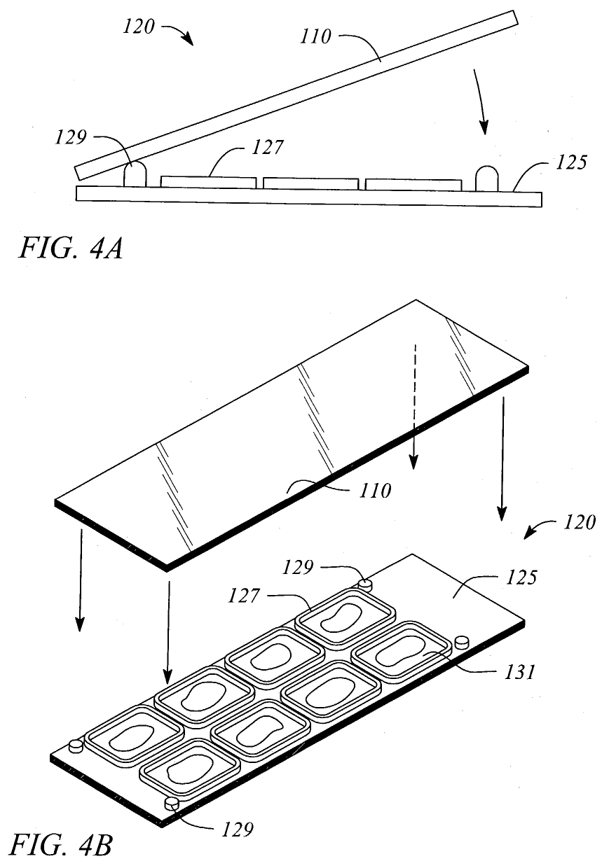

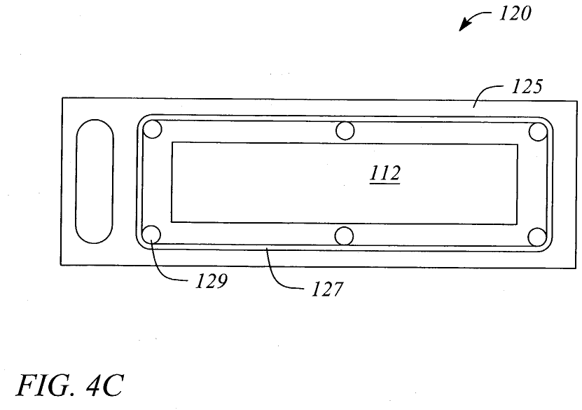

Referring now to FIGS. 4A-4C, the typical methods and apparatus of the present invention will now be described in more detail. An array hybridization apparatus 120 may comprise the first substrate 110 for holding the array 112, a second substrate 125 opposite the first substrate 110 for acting as a backing for the array hybridization apparatus 120, a gasket 127 interposed between the first substrate 110 and the second substrate 125, a fixed-height spacer to determine the distance between first substrate 110 and second substrate 125 (not shown), and a standoff 129. Standoff 129 is interposed between the first substrate 110 and the second substrate 125, adjacent to the gasket 127, wherein a uniform volume is defined between the first substrate 110, the second substrate 125, and the gasket 127 when the first substrate 110 and the second substrate 125 contact the gasket 127 and the spacer.

The hybridization apparatus 120 is designed for holding or positioning the array 112 so that maximum hybridizations/annealing of nucleic acids can take place between the array 112 and a target 114 of interest (target not shown in drawings).

The first substrate 110 may typically contain or be attached to the array 112 and may comprise any number of transparent or opaque materials such as glass, plastic, or other materials known in the art to contain or capable of containing arrays. First substrate 110 can be thought of as the array substrate, but need not contain the array 112. The array 112 could also be attached or part of the second substrate 125. The first substrate 110 may be designed in a variety of shapes, sizes and widths.

The second substrate 125 may typically comprise a material such as glass, plastic, silicon or other materials know in the art. The substrate 125 may be thought of as being the backing for the hybridization apparatus 120. However, in certain embodiments the substrate 125 may actually contain or comprise the array 112. The substrate 125 may be designed in a variety of shapes, sizes and widths.

The gasket 127 may be attached to the first substrate 110, or the second substrate 125 and is designed for holding or retaining the hybridization solutions for the array 112. It can also be added as a separate component, not firmly attached to either substrate. Typically, the gasket 127 will be rectangular in shape and will be attached to the substrate 125. The shape and design of the gasket 127 is not important to the invention.

FIG. 4A shows a side elevation view of the present invention. The figure shows a variety of gaskets 127 and how the first substrate 110 engages the second substrate 125 by way of the standoff 129. The standoff 129 is designed to be taller than the gasket 127; this prevents the first substrate 110 from contacting, spilling and spreading the solution held by the gasket 127 until first substrate 110 is positioned on all standoffs. The first substrate 110 may initially contact the standoff 129 at an angle; planar alignment is not required. In other words the standoff 129 allows the first substrate 110 to contact the second substrate 125 at any desired angle without spreading or smearing the solution volume held by the gasket 127.

The gasket 127 maintains a sufficient compressibility so as to form a seal between the first substrate 110, the gasket 127 and the second substrate 125 when they contact each other. The gasket 127 must retain the hybridization solution when the first substrate 110, substrate 125, and the gasket 127 contact each other. The gasket 127 may comprise any number of materials that are substantially deformable. For instance, the gasket 127 may comprise materials such as natural rubber, ethylene propylene rubbers, silicone, fluorocarbons, polyurethanes, acrylonitrile huna n's, neoprene, polyacrylates, buna S, fluorosilicones, and other non-synthetic and synthetic polymers.

The standoff 129 is important to the invention and several may be attached to the first substrate 110, the second substrate 125 or both. The standoff 129 may be used to guide the first substrate 110 to the second substrate 125 after the first substrate 110 has contacted the standoff 129. The first substrate 110 generally contacts the second substrate 125 at an angle and then is allowed to form a seal between the first substrate 110, the gasket 127 and the second substrate 125. Typically, the standoff 129 will be attached to the second substrate 125 when the gasket 127 is attached to the second substrate 125. The standoff 129 may comprise any number of shapes and sizes. It is important to the invention that the standoff 129 be taller in height on the second substrate 125 than the gasket 127. It may also be positioned in any number of positions on the first substrate 110 or second substrate 125 and may comprise compressible materials such a rubber, silicone, other polymeric materials, etc. This allows the gasket 127 to act as a seal and deform to the extent and height of the fixed height spacer (not shown). The fixed height spacer can range in height of from 25 to 500 microns, which also determines the final thickness of gasket 127 after it is compressed. This forms the uniform hybridization chamber 131 having a fixed volume. The standoff 129 will retain a height slightly larger than that of uncompressed gasket 127 in the range of from 40 to 1000 microns.

FIGS. 4B and 4C show two additional embodiments of the present invention. The gasket 127 and the standoff 129 have been mounted or attached to the second substrate 125. The gasket 127 helps to form the array hybridization chamber 131. As mentioned one or more standoffs 129 may be used and the standoff 129 may have a variety of shapes and sizes. FIG. 4A and 4C show two types of standoffs 129 (rectangular and round). Typically, as shown, the standoffs 129 are positioned adjacent to the gasket 127. They may be positioned anywhere convenient on the first substrate 110 or the second substrate 125, as long as they are far enough apart to hold the substrates apart farther than the uncompressed height of gasket 127. FIG. 4B shows the standoff 129 exterior to the gasket 127.

FIG. 4C shows another embodiment of the invention. In this embodiment the standoff 129 is used to position gasket 127. In this case, the gasket 127 may comprise a deformable material or substantially deformable material that contacts or is part of the exterior of the standoff 129. The standoff 129 maintains the gasket 127 under tension (See FIG. 4C) to form the hybridization chamber 131.

Although each of the embodiments are described separately, it could be imagined that various standoffs 129 could be placed on a substrate and then used according to the design desired or gaskets 127 that are employed. The standoffs, therefore, provide a significant and unexpected advantage that a variety of arrays and array chambers can be constructed that prevent spilling and allow for ease of loading on a single substrate. This provides added benefits and properties of flexibility in construction and use. For example, this embodiment of the invention provides more options for automation of the filling and assembly operation, since the components can be completely assembled before any hybridization liquid contacts the array 112. This allows conveyorized assembly for several to many units to take place before the actual hybridization reaction occurs.

These examples are for illustrative purposes only and the invention should not be interpreted to be limited to these embodiments only. The uniform array hybridization chamber 131 contains the array 112 and nucleic acids in a hybridization solution that is retained by the gasket 127. The uniform array hybridization chamber 131 provides for protection of the array and reproducibility of results.

Having described the apparatus of the invention, a description of the method of assembling or making the array hybridization apparatus is now in order.

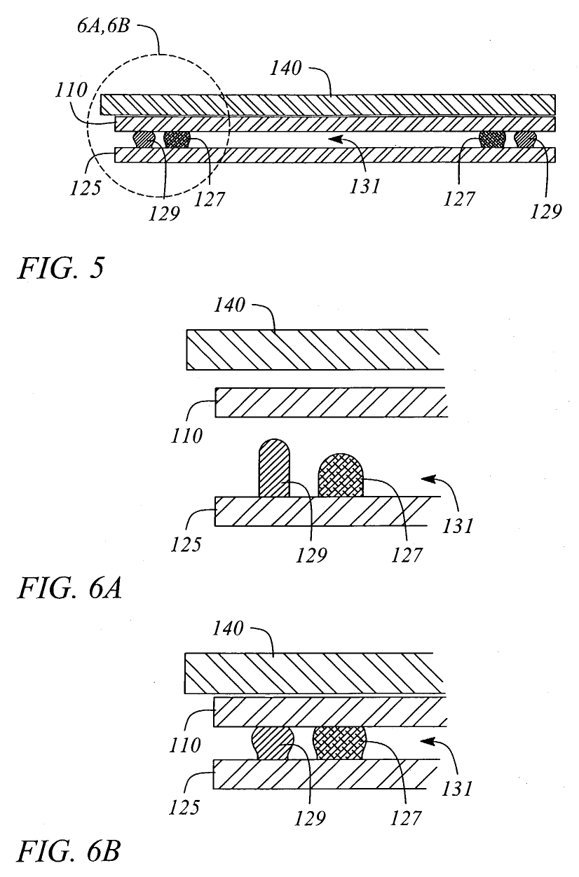

The array hybridization apparatus 120 can be easily assembled relative to other devices that contain fixed components. In its simplest form the array hybridization apparatus 120 may be constructed by providing the first substrate 110, the second substrate 125, the gasket 127 and the standoff 129 and then contacting each of these components to define the uniform array hybridization chamber 131 (See FIGS. 5, and 6A-6B). Typically, the gasket 127 and the standoff 129 are interposed between the first substrate 110 and the second substrate 125. This may be accomplished by first attaching the gasket 127 to either of the substrates, preferably second substrate 125. Next the array solutions are added to the gasket 127. The standoff 129 may then be added, or is already attached or constructed on the second substrate 125. The first substrate 110 with array or arrays 112 may then be placed onto the standoffs 129, ready to form the array hybridization chamber 131. The components may then be contacted by clamping or by joining the first substrate 110, a cover 140 (shown in FIGS. 5, 6A, 6B) and the second substrate 125 in any of a number of manners that are well known in the art. The components are contacted under sufficient, but not excessive pressure to form a sealed hybridization chamber 131 between the first substrate 110, the gasket 127 and the second substrate 125. FIGS. 6A and 6B show the importance of having a compressible standoff 129 and gasket 127 so that a tight seal can be formed between the first substrate 110, second substrate 125, and gasket 127 when the cover 140 is clamped onto these components.

Clearly, minor changes may be made in the form and construction of the invention without departing from the scope of the invention defined by the appended claims. It is not, however, desired to confine the invention to the exact form herein shown and described, but it is desired to include all such as properly come within the scope claimed.

We claim:

1. An array hybridization apparatus, comprising:
(a) a first substrate for holding an array;
(b) a second substrate opposite the first substrate for acting as a backing for the array hybridization apparatus;
(c) a gasket for holding an array solution;
(d) a standoff adjacent to the gasket and interposed between the first substrate and the second substrate for maintaining a space between the gasket and first substrate.

2. An array hybridization apparatus as recited in claim 1, wherein the height of the standoff is greater than the height of the gasket.

3. An array hybridization apparatus as recited in claim 1, wherein the standoff comprises a deformable material.

4. An array hybridization apparatus as recited in claim 1, wherein the standoff is attached to the first substrate.

5. An array hybridization apparatus as recited in claim 1, wherein the standoff is attached to the second substrate.

6. An array hybridization apparatus as recited in claim 1, wherein the standoff is attached to both the first substrate and the second substate.

7. An array hybridization apparatus as recited in claim 1, wherein the compressible or flexible standoff comprises a material selected from the group consisting of natural rubber, ethylene propylene rubbers, silicone, fluorocarbons, polyurethanes, acrylonitrile buna n's, neoprene, polyacrylates, buna S, and fluorosilicones.

8. An array hybridization apparatus as recited in claim 1, wherein the standoff is between 40 to 1000 microns in height.

9. A method of aligning an array hybridization apparatus comprising:
(a) providing a first substrate, second substrate and at least 2 standoffs; and
(b) contacting the first substrate to the standoff to align the array hybridization apparatus.

10. A method of making an array hybridization apparatus with a standoff for alignment, comprising:
(a) providing a first substrate opposite a second substrate;
(b) interposing a standoff between a first substrate and a second substrate;
(c) contacting the first substrate and second substrate to the standoff.

11. A method of making an array hybridization apparatus for aligning a gasket, comprising:
(a) providing a first substrate;
(b) attaching at least two standoffs to the first substrate;
(c) using the standoff to align at least one gasket on the first substrate using the standoff and the gasket.
